(19) United States
(12) Patent Application Publication
(10) Pub. No.: US 2018/0182476 A1
(43) Pub. Date: Jun. 28, 2018

(54) MAPPING OF CLINICAL FINDINGS IN FUNDUS IMAGES TO GENERATE PATIENT REPORTS

(71) Applicant: Carl Zeiss Meditec, Inc., Dublin, CA (US)

(72) Inventor: T. C. GANESH BABU, Bangalore (IN)

(21) Appl. No.: 15/835,311

(22) Filed: Dec. 7, 2017

(30) Foreign Application Priority Data
Dec. 23, 2016 (IN) 201611044135

(51) Int. Cl. G16H 15/00; G06F 3/0482; G16H 30/20; A61B 3/12

(52) U.S. Cl. CPC G16H 15/00; G06F 3/0482; G16H 40/63; A61B 3/12; G16H 30/20

(57) ABSTRACT

An improved method of generating a fundus image screening report of a patient is described. The method includes displaying a fundus image to a reviewer. A prepopulated list of clinical findings relating to one or more areas in the fundus image is displayed to the reviewer. An input from the reviewer is received selecting one or more clinical findings from the prepopulated list. A report containing plausible clinical observations and recommendations is generated based on the selected clinical findings, and the generated report is displayed or stored for a further processing thereof.

### TECHNICAL FIELD

The present application generally relates to generating a fundus image screening report of a patient using an improved report generatation process, which is more simplified, accurate, and time efficient.

### BACKGROUND

When a fundus image reader/clinician checks findings in an image, he/she has to further analyze these findings before concluding on clinical observations and optimal recommendations for the noted findings. Manually performing and keying in the clinical observations and recommendations for reporting is a laborious process and sometimes contradicts with the noted findings in the image resulting in a conflicting final patient report.

Presently, the image readers/clinicians refer the cases which they are unsure of to an expert (e.g., an eye specialist or doctor) who reviews such cases and writes the necessary observations and/or recommendations on behalf of the clinicians. Even if these observations and/or recommendations are provided by the image reader, the expert still has to spend an adequate amount of time reviewing and making sure that they are clinically related to the noted findings in the image and are valid for providing it to the patients. The limitations of this process is that—it is laborious, time intensive, depends on the availability of an expert for referral, and there is a likelihood of conflicting observation when not scrutinized by the expert.

Here we describe a new and unique approach for mapping clinical findings in fundus images to generate fundus image screening reports that overcomes the limitations of the existing approaches.

### SUMMARY

It is an object of the present invention to improve the existing process of generating medical reports by mapping the findings noted in a given image (e.g., fundus image) to automatically provide logical and relevant clinical observations with appropriate recommendations for review by a trained image reader/clinician (herein referred to as a reviewer) in an editable text format. The reviewer can review these automatically generated clinical observations and recommendations and revise the same as per his/her judgment before finalizing a patient report. To prompt the clinical observations with recommendations, the technique of the present invention works on a clinical logic based on published medical literature and guidelines for diagnosis/management of eye diseases by accredited ophthalmology associations, as well on clinical acumen of key opinion leaders in ophthalmology.

This technique of report generation is advantageous in a number of respects. For instance, it 1) simplifies the overall workflow of report creation, 2) prepares a full patient report, using the clinical findings noted by a reviewer, in compliance with the published medical literature and guidelines for diagnosis/management of eye diseases, 3) reduces the reviewer errors related to conflicting observations/recommendations, 4) reduces the turnaround time to report on a fundus image, and 5) conserves the time involvement by an expert or eye specialist doctor to finalize the report. It should be understood that the foregoing advantages are provided by way of example and other advantages and/or benefits are also possible and contemplated.

It should be noted that the language used in the specification has been principally selected for readability and instructional purposes and not to limit the scope of the inventive subject matter.

### BRIEF DESCRIPTION OF THE FIGURES

FIGS. 1A and 1B illustrate two prior art fundus imaging system designs that may be used to capture fundus images for use with the present invention. In particular, FIG. 1A illustrates an external illumination design. FIG. 1B illustrates an internal illumination design.

FIG. 2 is a flowchart of an example method for generating a fundus image screening report of a patient according to one aspect of the present invention.

FIG. 3A is one example of a graphical user interface (GUI) for displaying a prepopulated list of clinical findings to a reviewer that may be selected to automatically generate an initial fundus image screening report of a patient.

FIG. 3B is another example of a GUI for displaying a prepopulated list of clinical findings to a reviewer that may be selected to automatically generate an initial fundus image screening report of a patient.

FIG. 3C is a GUI displaying plausible clinical observations and recommendations generated based on the clinical findings selected by the reviewer in the GUIs of FIGS. 3A and 3B.

FIGS. 4A, 4B and 4C show an example fundus image screening report of a patient according to one aspect of the present invention.

FIG. 5 is a block diagram of a general computer system that may perform the functions discussed in the disclosure according to one aspect of the present invention.

### DETAILED DESCRIPTION

All patent and non-patent references cited within this specification are herein incorporated by reference in their entirety to the same extent as if the disclosure of each individual patent and non-patient reference was specifically and individually indicated to be incorporated by reference in its entirely.

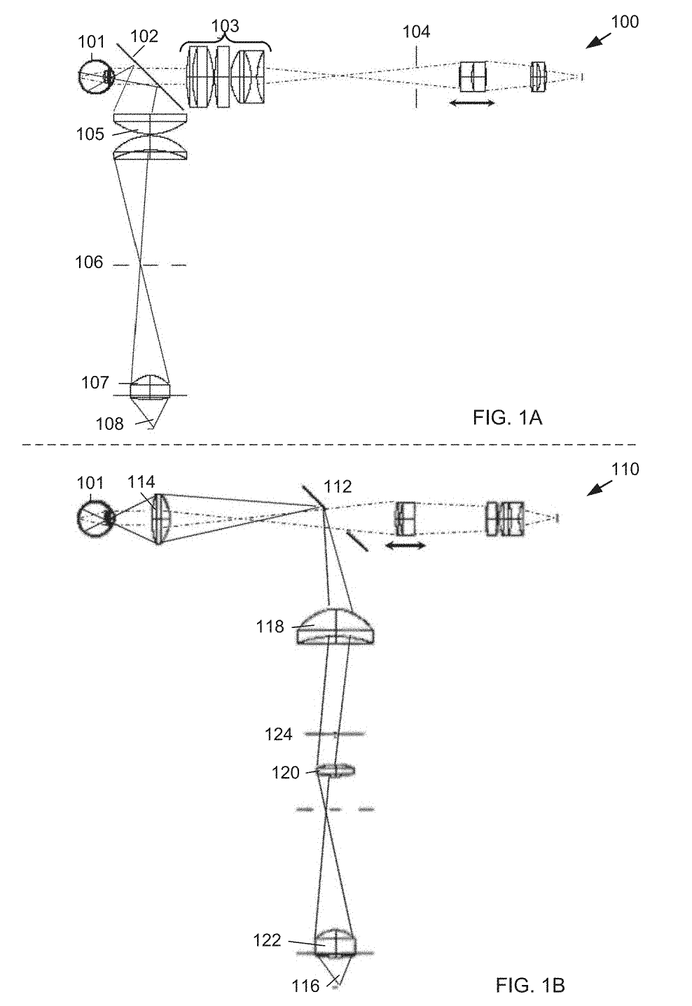

FIGS. 1A and 1B illustrate elements of two typical prior art fundus imaging systems (see for example, DeHoog, E. and J. Schwiegerling (2009). “Fundus camera systems: a comparative analysis.” Appl. Opt. 48(2): 221-228). It should be understood that the fundus imaging systems of FIGS. 1A and 1B are just two exemplary systems that can be used with the various embodiments discussed in the present application. However, this is not limiting and other ophthalmic diagnostic/imaging systems are also possible for use with these embodiments and are within the scope of the present disclosure. For instance, an optical coherence tomography (OCT) system can be used to obtain eye images that could be evaluated with the embodiments of the present invention. Also, it should be noted a device capable of taking fundus images for use with the present invention can either be stationary (i.e., fixed at one location) or portable (e.g., handheld) device, and may be connected to a network (e.g., internet) for communication with other systems/servers/devices. Furthermore, such a device may either store data (e.g. fundus images) internally or may store the data on a cloud for later access and/or retrieval by other devices or systems. In some instances, the device may even serve as a self-kiosk system that a subject can use to acquire his/her eye image, which can then be sent to the cloud for evaluation.

FIG. 1A illustrates an external illumination design 100 using a beam splitter 102 placed in front of the eye 101 to combine the illumination and imaging paths. A source 108 is imaged onto the pupil of the eye by lenses 105 and 107. An annulus 106 is placed between the lenses and positioned conjugate to the pupil of the eye. FIG. 1B illustrates an internal illumination design 110 that uses a mirror 112 with a central hole placed conjugate to the pupil of eye 101 to combine the illumination and imaging paths. In FIG. 1B, the fact that the illumination and imaging paths share the objective lens 114 requires a more complicated system to eliminate back reflections. The source 116 is reimaged to the mirror by lenses 118, 120, and 122. Back reflections from the front and back surfaces of the objective are removed by a black dot 124 placed conjugate to the back surface of the objective. The internal system 110 of FIG. 1B uses a single aspheric objective 114 to reduce the number of surfaces contributing to the back reflections, whereas the objective 103 in the external system 100 of FIG. 1A can consist of multiple elements because it is not part of the illumination pathway and will not contribute to back reflections. The only difference in the imaging paths of both systems is the existence of a baffle 104 in FIG. 1A. This baffle, placed conjugate to the pupil of the eye, helps reduce corneal back reflections and limits the entrance pupil diameter of the imaging system. In the internal system 110, the mirror 112 with the central hole serves the same purpose as the baffle 104 in the external system 100. An iris can be placed immediately behind the mirror with the central hole to allow for further control of the entrance pupil diameter and for elimination of back reflections.

### Automatic Report Generation Workflow

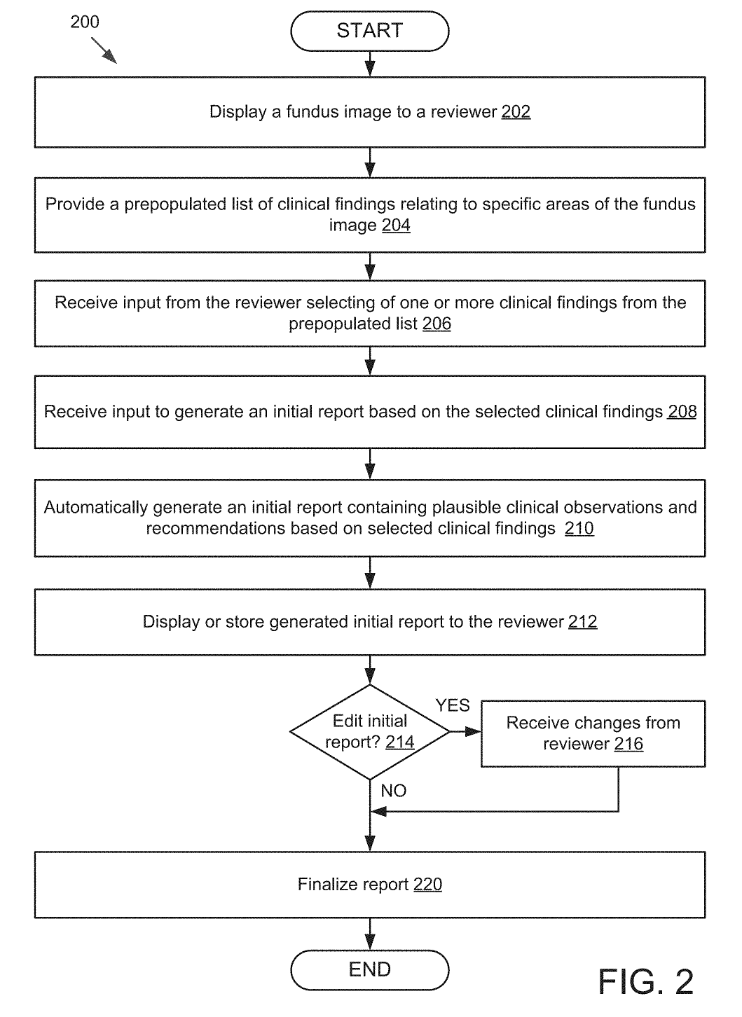

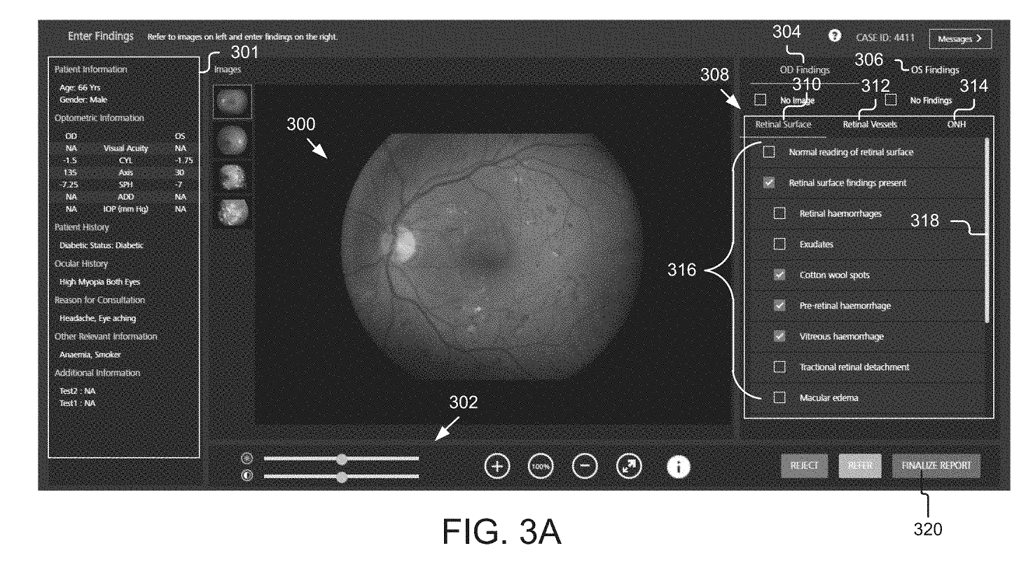

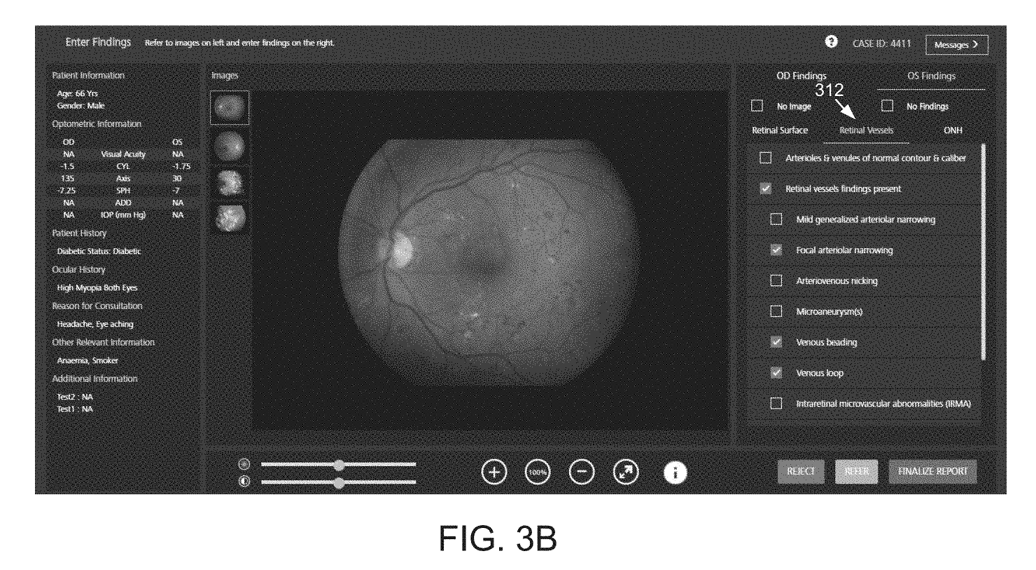

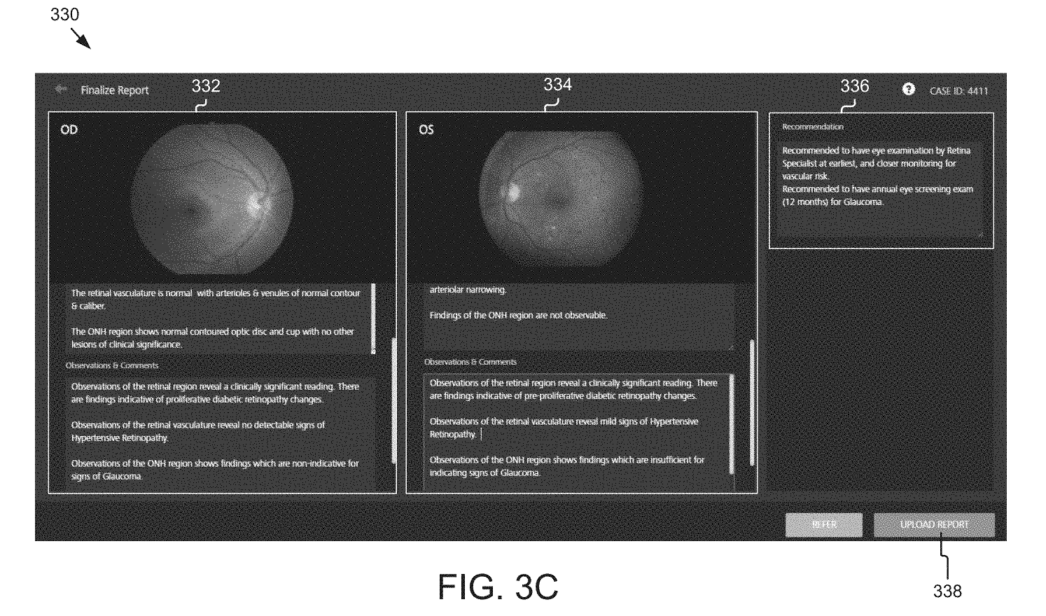

FIG. 2 shows an example method 200 for generating a fundus image screening report of a patient. The method 200 begins by displaying a fundus image to a reviewer. The reviewer may be a person who obtained the fundus image by himself/herself (i.e., device operator), a person who is skilled in examining fundus images of patients (i.e., eye specialist/doctor), or a person who is trained in making an initial analysis/examination on the fundus images but a final review is required to be performed by the eye specialist/doctor for finalizing a patient's fundus image screening report. The reviewer may be located either locally (i.e., where the fundus image is obtained) or at a remote site where the remote site is designated for remotely examining the fundus images and then sending the results of the examination (e.g., clinical observations, recommendations, etc.) to either eye specialists/doctors for further review/examination or directly to patients with which the examined fundus images are associated. The fundus image can be obtained, for example, using the fundus imaging system 100 or 110 illustrated in FIG. 1. An example of such a fundus image is shown in FIG. 3A by reference numeral 300. Next, in block 204, a prepopulated list of clinical findings relating to specific areas in the fundus image is provided to the reviewer. These areas in the image are, for example, related to retinal surface, retinal vessels, and optic nerve head (ONH). It should be noted that clinical findings relating to other areas in a fundus image are possible and are within the scope of the present disclosure.

By way of an example and without limitation, some of the clinical findings related to retinal surface, retinal vessels, and ONH which may be provided to a reviewer in a prepopulated list are as follows:

| Retinal Surface | Retinal Vessels | Optic Nerve Head (ONH) |
|---|---|---|
| Retinal surface not | Retinal vessels not | ONH not observable |
| observable | observable | ONH normal reading |
| Normal reading of retinal | Arterioles & venules of | ONH findings present |
| surface | normal contour & caliber | Rim loss |
| Retinal hemorrhage | Focal anteriolar narrowing | Retinal nerve fiber layer |
| Exudates | Microaneurysm(s) | (RNFL) loss |
| Cotton wool spots | Intraretinal microvascular | Optic nerve cupping |
| Macular edema | abnormalities (IRMA) | Focal notching |
| Drusen | Neovascularization | Optic disc hemorrhage |
| Other lesions | Fibrovascular changes | Optic disc edema |

In block 206, an input from the reviewer is received selecting one or more clinical findings from the prepopulated list that was presented to the reviewer. For instance, with respect to FIG. 3A, the reviewer for making clinical findings relating to retinal surface in the image, the reviewer can select desired options in the prepopulated list 310 (e.g., using check boxes as indicated by reference numeral 316) for which he/she wants to make observations, recommendations, and/or any comments. Similarly, if the reviewer wants to make clinical findings relating to retinal vessels in the image, then the reviewer can select desired options in the prepopulated list 312 as shown in FIG. 3B.

Making clinical findings in an image through a prepopulated list as discussed above is advantageous because 1) it reduces time and effort for a reviewer when making such findings since the possible findings are premade and the reviewer just needs to pick/select those which apply to the image under consideration, 2) plausible clinical observations and/or recommendations are predetermined for these possible findings, which enables the reviewer to quickly generate a medical report for a patient containing the clinical observations and/or recommendations in an automated manner (discussed in more detail below with respect to block 210), 3) allows even a novice reviewer to make a report for a patient since the chances of error or inaccuracies associated with such an automated process is minimal, 4) requires minimum or no changes by an expert or eye specialist/doctor when reviewing or finalizing the report prepared by the reviewer.

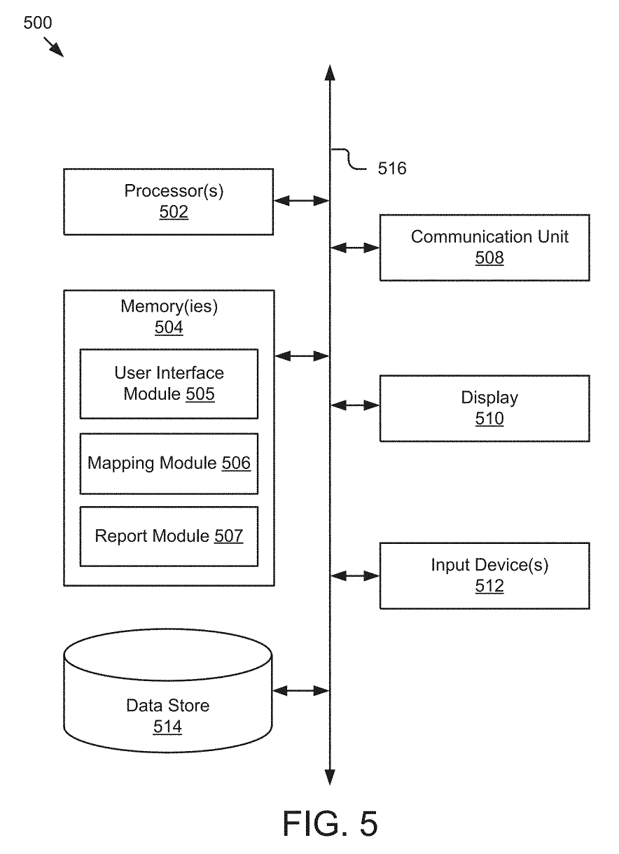

In block 210, based on the clinical findings selected by the reviewer as discussed above, an initial report containing plausible clinical observations and recommendations is automatically generated. Automatic as used here, means that a system (e.g., a general purpose computing device such as the computing device 500 shown in FIG. 5), without human intervention, generates a report containing clinical observations and recommendations based on the reviewer selected clinical findings. The system may generate this report based on a mapping of each of the clinical findings to its possible observation and recommendation. Such a mapping may be pre-prepared by or in collaboration with an expert (e.g., an eye specialist/doctor) and stored in a data store, such as the data store 514 for real-time access and/or retrieval when a reviewer is examining a fundus image and preparing a report.

Following shows an example mapping of some clinical findings (relating to retinal surface) to possible observations upon examining a fundus image and recommendations that may be suggested by an eye specialist:

| Clinical Finding | Possible Clinical Observation | Possible Recommendation |
|---|---|---|
| 1. Normal reading of retinal surface | No findings indicative of any | Recommended to have annual eye |
| | changes in the retinal surface | screening exam (12 months) for |
| | | diabetic retinopathy. |
| 2. (Retinal hemorrhages or Exudates | There are findings indicative of | Recommended to have eye |
| or pre-retinal hemorrhage or vitreous | diabetic retinopathy, maculopathy, | examination by a retina |
| hemorrhage) and (Macular edema) | and photocoagulation changes. | specialist as |
| and (Focal/grid laser scars or | | soon as possible. |
| peripheral scatter laser scars) | | |
| 3. (#2) and (Drusen) | There are findings indicative of | Recommended to have eye |
| | diabetic retinopathy, maculopathy, | examination by a retina |
| | and photocoagulation changes with | specialist as |
| | early AMD changes. | soon as possible. |
| 4. (#2) and (other lesions) | There are findings indicative of | Recommended to have eye |
| | diabetic retinopathy, maculopathy, | examination by a retina |
| | and photocoagulation changes with | specialist as |
| | other non-specific changes in the | soon as possible. |
| | retinal region. | |
| 5. (#2) and (Drusen) and (other | There are findings indicative of | Recommended to have eye |
| lesions) | diabetic retinopathy, maculopathy, | examination by a retina |
| | photocoagulation, and early AMD | specialist as |
| | changes with other non-specific | soon as possible. |
| | changes in the retinal region. | |

In block 212, the initial report automatically generated by the system, based on the mapping technique discussed above, is displayed to the reviewer (see for example FIG. 3C). In block 214, the reviewer, before finalizing the report, decides whether there is a need to make any changes to the clinical observations and/or recommendations. If there is, then the reviewer changes are received (block 216) and the report is finalized based on those changes (block 220). Otherwise, the method 200 just proceeds to block 220 to finalize the report. The finalized report can either be sent directly to the patient with whom the fundus image was associated, an eye expert/specialist/doctor for further review, or uploaded on a central server/cloud where the report is saved for later retrieval and/or access by the eye expert or the reviewer. The method 200 then terminates and ends.

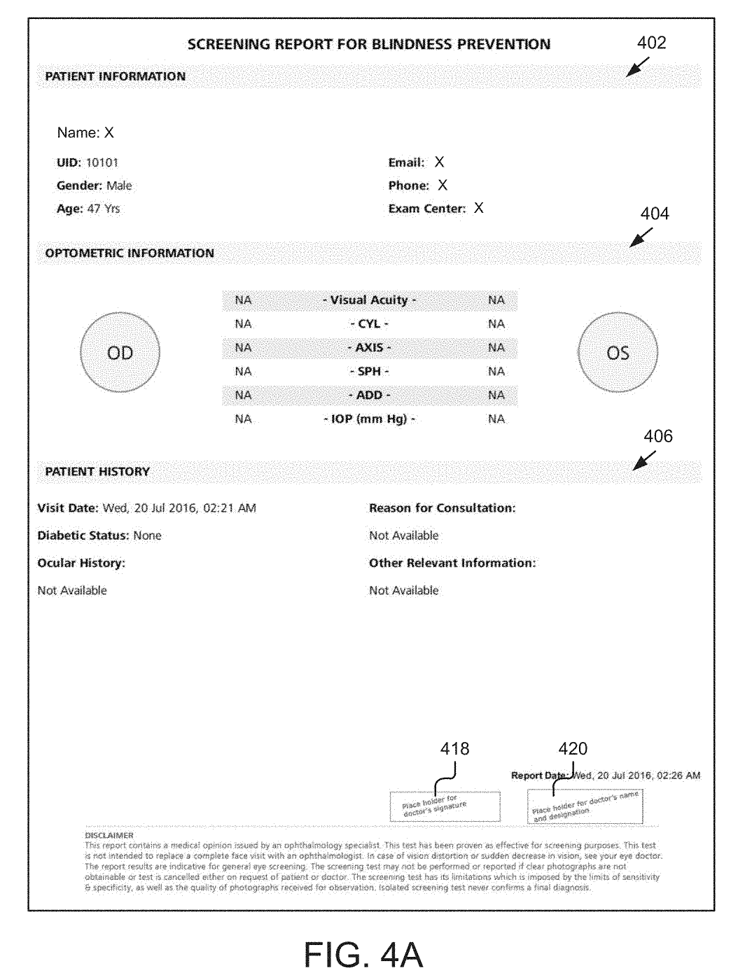

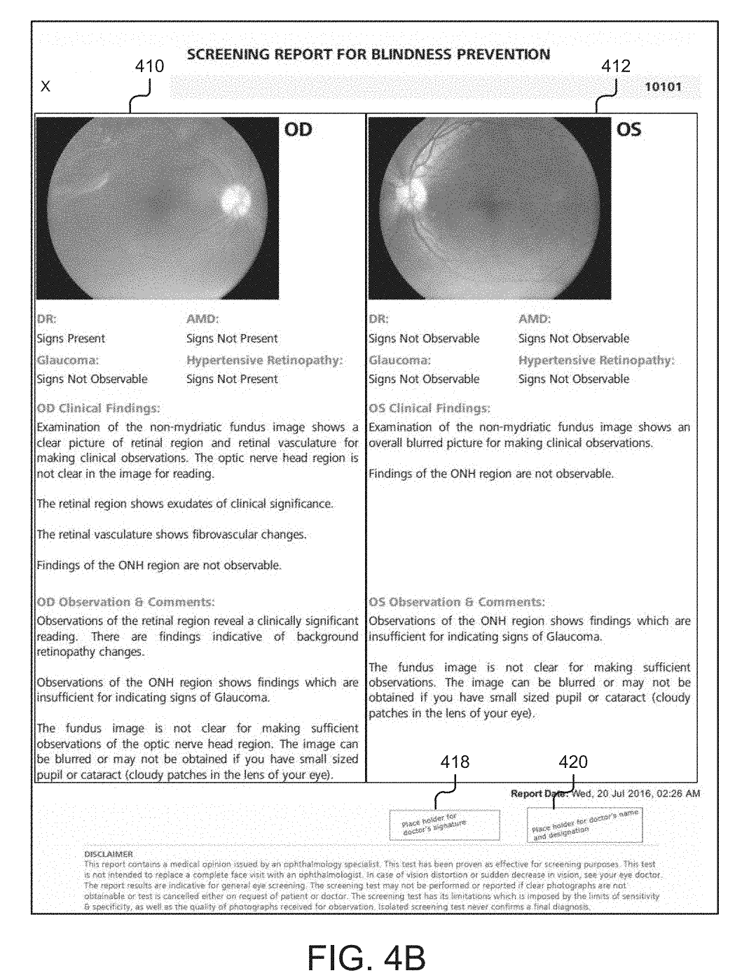

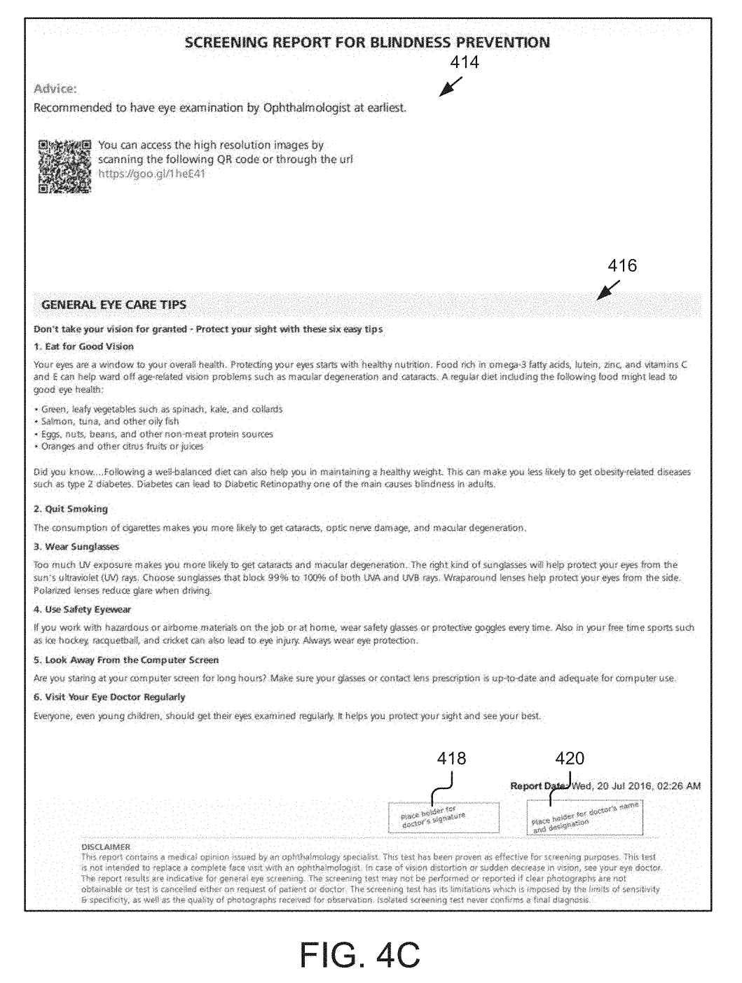

FIG. 3A shows an example user interface which a reviewer can use to make clinical findings for a fundus image 300. The left portion 301 of the interface can be used to enter patient information for which the fundus image 300 is being examined. The patient information may include, for example, patient's name, age, optometric information for each eye, history, ocular history, reasons for consultation, and any other relevant or additional information. This patient information will be included in a final fundus image screening report of the patient as shown for example in FIG. 4A. Reference numeral 300 shows the fundus image of the patient, which may be obtained through the fundus imaging system 100 or 110. The image 300 can be optimized (adjust brightness, contrast, etc.) using the scroll bars 302. The reviewer can make clinical findings for the left eye or the right eye of the patient by switching between the tabs 304 and 306, respectively. Reference numeral 308 shows a prepopulated list of clinical findings available for the retinal surface 310, retinal vessels 312, and optic nerve head 314. Specifically, the prepopulated list 308 in FIG. 3A shows clinical findings relating to the retinal surface 310. FIG. 3B shows the prepopulated list of clinical findings relating to the retinal vessels 312. The reviewer can make findings in the fundus image 300 by switching between these tabs (310-314) and selecting one or more checkboxes 316 underneath a selected tab. In case the prepopulated list for a selected tab is long, the reviewer can scroll through the list and see all the available options using the scroll bar 318. Once the reviewer has made all the necessary clinical findings in the fundus image 300, he/she may choose to generate a report based on those findings by clicking the “finalize report” button 320.

FIG. 3C shows an example user interface 330 which is generated based on the clinical findings selected by the reviewer using the interface depicted, for example, in FIG. 3A or 3B. In particular, the interface 300 is generated once the reviewer has check boxed all the desired clinical findings in a fundus image and opted to generate a report by clicking on the finalize report button 320 (FIG. 3A). The interface 300 shows observations and any comments for the right eye of the patient (indicated by reference numeral 332), observations and any comments for the left eye (indicated by reference numeral 334), and possible recommendations 336 based on the selected clinical findings and/or observations. The observations and recommendations shown in the interface 330 are automatically generated based on the mapping technique discussed herein. The reviewer viewing the interface 330 may make any necessary adjustments (e.g., add/delete) in the automatically generated observations 332 and/or 334 and the recommendations 336. Once the observations and recommendations are final, the reviewer may choose to upload the report for further analysis or processing (e.g., expert's review) by clicking on the “upload report” button 338.

FIGS. 4A-4C shows an example fundus image screening report of a patient, which can be generated using the interfaces depicted in FIGS. 3A-3C. In particular, the first page of the report (FIG. 4A) includes general patient information including the patient information 402, optometric information 404 associated with each eye of the patient, and patient history 406. The second page of the report (FIG. 4B) includes clinical findings noted by a reviewer, his/her observations, and any comments for the right eye (indicated by reference numeral 410) and the left eye (indicated by reference numeral 412) of the patient. The last page of the report (FIG. 4C) shows recommendation(s) 414 to obviate or mitigate signs of any eye diseases (e.g., diabetic retinopathy (DR), glaucoma, age related macular degeneration (AMD), hypertensive retinopathy, etc.) noted based on the clinical findings and/or observations made in page 2 of the report (FIG. 4B). The last page also includes some general eye care tips 416 for the overall healthiness of the eye. Each of the report pages (FIGS. 4A-4C) includes placeholders 418 and 420 for entering the doctor's name and signature.

### Example Computer System

FIG. 5 is a block diagram of a general computer system 500 that may perform the functions discussed in this disclosure according to one aspect of the present invention. The computer system 500, as depicted, may include one or more processors 502, one or more memories 504, a communication unit 508, a display 510, one or more input devices 512, and a data store 514.

The components 502, 504, 508, 510, 512, and 514 are communicatively coupled via a communication or system bus 516. The bus 516 can include a conventional communication bus for transferring data between components of a computing device or between computing devices. It should be understood that the computing system 500 described herein is not limited to these components and may include various operating systems, sensors, video processing components, input/output ports, user interface devices (e.g., keyboards, pointing devices, displays, microphones, sound reproduction systems, and/or touch screens), additional processors, and other physical configurations.

The processor(s) 502 may execute various hardware and/or software logic, such as software instructions, by performing various input/output, logical, and/or mathematical operations. The processor(s) 502 may have various computing architectures to process data signals including, for example, a complex instruction set computer (CISC) architecture, a reduced instruction set computer (RISC) architecture, and/or architecture implementing a combination of instruction sets. The processor(s) 502 may be physical and/or virtual, and may include a single core or plurality of processing units and/or cores. In some embodiments, the processor(s) 502 may be capable of generating and providing electronic display signals to a display device, such as the display 510, supporting the display of images, capturing and transmitting images, performing complex tasks including various types of feature extraction and sampling, etc. In some embodiments, the processor(s) 502 may be coupled to the memory(ies) 504 via a data/communication bus to access data and instructions therefrom and store data therein. The bus 516 may couple the processor(s) 502 to the other components of the computer system 500, for example, the memory(ies) 504, the communication unit 508, or the data store 514.

The memory(ies) 504 may store instructions and/or data that may be executed by the processor(s) 502. In the depicted embodiment, the memory(ies) 504 stores at least a user interface module 505, a mapping module 506, and an automatic report generation module 507, each of which may include software, code, logic, or routines for performing any and/or all of the techniques described herein. For instance, the user interface module 505 may generate the user interfaces as depicted in FIGS. 3A-3C, which a reviewer may use to make clinical findings in a fundus image; the mapping module 506 may perform the automatic mapping of the clinician's findings with the appropriate clinical observations and recommendations (predetermined and stored in the data store 514); and the report module 507 may generate the final report as shown, for example, in FIGS. 4A-4C. In some embodiments, the memory(ies) 504 may also be capable of storing other instructions and data including, for example, an operating system, hardware drivers, other software applications, databases, etc. The memory(ies) 504 are coupled to the bus 516 for communication with the processor(s) 502 and other components of the computer system 500. The memory(ies) 504 may include a non-transitory computer-usable (e.g., readable, writeable, etc.) medium, which can be any apparatus or device that can contain, store, communicate, propagate or transport instructions, data, computer programs, software, code, routines, etc. for processing by or in connection with the processor(s) 502. A non-transitory computer-usable storage medium may include any and/or all computer-usable storage media. In some embodiments, the memory(ies) 504 may include volatile memory, non-volatile memory, or both. For example, the memory(ies) 504 may include a dynamic random access memory (DRAM) device, a static random access memory (SRAM) device, flash memory, a hard disk drive, a floppy disk drive, a CD ROM device, a DVD ROM device, a DVD RAM device, a DVD RW device, a flash memory device, or any other mass storage device known for storing instructions on a more permanent basis.

The computer system 500 may include one or more computers or processing units at the same or different locations. When at different locations, the computers may be configured to communicate with one another through a wired and/or wireless network communication system, such as the communication unit 508. The communication unit 508 may include network interface devices (I/F) for wired and wireless connectivity. For example, the communication unit 508 may include a CAT-type interface, USB interface, or SD interface, transceivers for sending and receiving signals using Wi-Fi™; Bluetooth®, or cellular communications for wireless communication, etc. The communication unit 508 can link the processor(s) 502 to a computer network that may in turn be coupled to other processing systems.

The display 510 represents any device equipped to display electronic images and data as described herein. The display 510 may be any of a conventional display device, monitor or screen, such as an organic light-emitting diode (OLED) display, a liquid crystal display (LCD). In some embodiments, the display 510 is a touch-screen display capable of receiving input from one or more fingers of a user. For example, the device 510 may be a capacitive touch-screen display capable of detecting and interpreting multiple points of contact with the display surface. The input device(s) 512 are any devices for inputting data on the computer system 500. In some embodiments, an input device is a touch-screen display capable of receiving input from one or more fingers of the user. The functionality of the input device(s) 512 and the display 510 may be integrated, and a user of the computer system 500 may interact with the system by contacting a surface of the display 510 using one or more fingers. In other embodiments, an input device is a separate peripheral device or combination of devices. For example, the input device(s) 512 may include a keyboard (e.g., a QWERTY keyboard) and a pointing device (e.g., a mouse or touchpad). The input device(s) 512 may also include a microphone, a web camera, or other similar audio or video capture devices.

The data store 514 can be an information source capable of storing and providing access to data. In the depicted embodiment, the data store 514 is coupled for communication with the components 502, 504, 508, 510, and 512 of the computer system 500 via the bus 516, and coupled, via the processor(s) 502, for communication with the user interface module 505, the mapping module 506, and/or the report module 507. In some embodiments, the mapping module 506 is configured to manipulate, i.e., store, query, update, and/or delete, data stored in the data store 514 using programmatic operations.

In the above description, for purposes of explanation, numerous specific details are set forth in order to provide a thorough understanding of the specification. It should be apparent, however, that the subject matter of the present application can be practiced without these specific details. It should be understood that the reference in the specification to “one embodiment”, “some embodiments”, or “an embodiment” means that a particular feature, structure, or characteristic described in connection with the embodiment is included in one or more embodiments of the description. The appearances of the phrase “in one embodiment” or “in some embodiments” in various places in the specification are not necessarily all referring to the same embodiment(s).

Furthermore, the description can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The foregoing description of the embodiments of the present subject matter has been presented for the purposes of illustration and description. It is not intended to be exhaustive or to limit the present embodiment of subject matter to the precise form disclosed. Many modifications and variations are possible in light of the above teaching. It is intended that the scope of the present embodiment of subject matter be limited not by this detailed description, but rather by the claims of this application. As will be understood by those familiar with the art, the present subject matter may be embodied in other specific forms without departing from the spirit or essential characteristics thereof. Furthermore, it should be understood that the modules, routines, features, attributes, methodologies and other aspects of the present subject matter can be implemented using hardware, firmware, software, or any combination of the three.

What is claimed is:

1. A method of generating a fundus image screening report of a patient, said method comprising:
displaying a fundus image to a reviewer;
displaying a prepopulated list of clinical findings relating to one or more areas in the fundus image;
receiving an input from the reviewer selecting one or more clinical findings from the prepopulated list;
generating a report containing plausible clinical observations and recommendations based on the selected clinical findings; and
displaying or storing the generated report or a further processing thereof.

2. The method as recited in claim 1, wherein the report generating step comprises:
mapping each of the selected clinical findings to a predetermined clinical observation and recommendation set for the clinical finding, wherein the predetermined clinical observations and recommendations are stored in a data store; and
populating the report with the predetermined clinical observations and recommendations for the selected clinical findings based on the mapping.

3. The method as recited in claim 2, further comprising:
receiving one or more changes from the reviewer to the generated report; and
updating the report based on the received changes.

4. The method as recited in claim 3, wherein a change includes an observation or a recommendation change.

5. The method as recited in claim 1, further comprising:
receiving an input from the reviewer to upload the report; and
uploading the report to a cloud server.

6. The method as recited in claim 1, wherein the one or more areas in the fundus image are retinal surface, retinal vessels, and optic nerve head.

7. The method as recited in claim 1, wherein the clinical observations and recommendations relate to the patient's eye disease condition.

8. The method as recited in claim 7, wherein the eye disease is diabetic retinopathy, age related macular degeneration, hypertensive retinopathy, and glaucoma.

9. The method as recited in claim 1, wherein the reviewer is a clinician, an image reader, a device operator, or an eye specialist.

10. The method as recited in claim 1, wherein the method is performed at a location that is remote from where the fundus image was collected.

11. A system for generating a fundus image screening report of a patient, said system comprising:
a display with a graphical user interface for displaying a fundus image and a prepopulated list of clinical findings relating to one or more areas in the fundus image to a reviewer;
an input device for receiving an input from the reviewer selecting one or more clinical findings from the prepopulated list; and
a processor for automatically generating a report containing plausible clinical observations and recommendations based on the selected clinical findings, and wherein said display is further configured for displaying the results of the generated report to the reviewer.

12. The system as recited in claim 11, wherein to generate the report, the processor is configured to:
map each of the selected clinical findings to a predetermined clinical observation and recommendation set for the clinical finding, wherein the predetermined clinical observations and recommendations are stored in a data store; and
populate the report with the predetermined clinical observations and recommendations for the selected clinical findings based on the mapping.

13. The system as recited in claim 12, wherein:
the input device is further configured to receive one or more changes from the reviewer to the generated report; and
wherein the processor is further configured to update the report based on the received changes.

14. The system as recited in claim 13, wherein a change includes an observation or a recommendation change.

15. The system as recited in claim 11, wherein:
the input device is further configured to receive an input from the reviewer to upload the final report; and
the processor is further configured to upload the final report to a cloud server.

16. The system as recited in claim 11, wherein the one or more areas in the fundus image are retinal surface, retinal vessels, and optic nerve head.

17. The system as recited in claim 11, wherein the clinical observations and recommendations relate to the patient's eye disease condition.

18. The system as recited in claim 17, wherein the eye disease is diabetic retinopathy, age related macular degeneration, hypertensive retinopathy, and glaucoma.

19. The system as recited in claim 11, wherein the reviewer is a clinician, an image reader, a device operator, or an eye specialist.

20. The system as recited in claim 11, wherein the system is located remotely from a fundus imaging system with which the fundus image was acquired.
